(19) United States
(12) Patent Application Publication
(10) Pub. No.: US 2015/0088247 A1
(43) Pub. Date: Mar. 26, 2015

(54) TISSUE-ENGINEERED HEART VALVE FOR TRANSCATHETER REPAIR

(71) Applicant: Cytograft Tissue Engineering, Inc., Novato, CA (US)

(72) Inventors: Nicolas L'Heureux, San Rafael, CA (US); Todd N. McAllister, San Anselmo, CA (US); Nathalie Dusserre, San Rafael, CA (US)

(73) Assignee: Cytograft Tissue Engineering, Inc., Novato, CA (US)

(21) Appl. No.: 14/387,323

(22) PCT Filed: Mar. 25, 2013
(86) PCT No.: PCT/US13/33768

§ 371 Date: Sep. 23, 2014

Related U.S. Application Data
(60) Provisional application No. 61/615,111, filed on Mar. 23, 2012.

(51) Int. Cl. A61F 2/24

(52) U.S. Cl. CPC A61F 2/2418; A61F 2/2427; A61F 2/2415; A61F 2210/0076
USPC 623/2.11; 623/2.14; 435/325; 435/366; 435/375

(57) ABSTRACT

A single-layer tissue sheet having a puncture strength of 2 kgf to 6 kgf. A valve, such as a heart valve, made of one or more leaflets formed from a single-layer tissue sheet. A method of making a tissue sheet having a puncture strength of 2 kgf to 5 kgf. The ultra-strong tissue sheets described herein have very long culture times, such as in excess of 20 weeks. Valves that comprise one or more leaflets made from the ultra-strong tissue sheets described herein may be delivered via trans-cathete aortic valve implantation.

### CLAIM OF PRIORITY

This application claims the benefit of priority under 35 U.S.C. §119(e) to U.S. provisional application Ser. No. 61/615,111, filed Mar. 23, 2012, which is incorporated herein by reference in its entirety.

### TECHNICAL FIELD

The technology described herein generally applies to the field of tissue engineering, repair of tissues damaged by cardiovascular disease, and endovascular approaches to such repairs. More specifically, the technology pertains to a valve made from tissue-engineered materials that can be delivered through open or percutaneous approaches, and methods of making the valve. The tissue-engineered valves can replace heart valves such as aortic valves, pulmonary valves, tricuspid valves, and mitral valves, as well as venous valves or stomach valves.

### BACKGROUND

The human body contains a number of different types of valves, any of which can fail due to disease or some genetic abnormality during a patient's lifetime. Of particular interest are heart valves, whose failure can lead to a patient's rapid demise.

One aspect of treatment of heart disease is the surgical replacement of one or more of the heart's four valves, any of which can become damaged or diseased. The human heart has four chambers, each of which is equipped with a one-way valve. The four valves are referred to variously as: aortic, pulmonary, mitral (or bicuspid), and tricuspid. The first two (also called semilunar valves) regulate blood flow through arteries that leave the heart, whereas the latter two (also called atrioventricular valves) control bloodflow into the heart. Each valve has a structure comprising an annular portion situated at the neck of a vessel where it joins the heart muscle, and two or three flaps, referred to (in native tissue) as cusps, that regulate the flow of blood through the annulus.

Replacement of a heart valve is usually via a valve implant. Most valve implants made today are used to replace failing heart valves but some valve implants are also being developed to replace valves found in veins. Valve implants can be made exclusively of synthetic material, in which case they are often referred to as “mechanical valves”. Synthetic valves have the limitation that they can accrete material over time and may also be thrombogenic. The lifespan of a valve implant is typically only 15 or so years, meaning that a younger patient may require two or more surgeries during their lifetime to replace the valve. As such surgeries tend to be expensive and complicated, longer-lived valve implants are desired.

Valve implants can also be made exclusively of biological tissue, or made of a combination of both synthetic and biological components. Valves made from biological tissues are often referred to as “tissue-valves”, and utilize tissues harvested from cadavers or animal sources. Tissue valves can be actual valves harvested from a cadaver of an animal, or a valve created from harvested tissues that were not originally present in valves. Such created tissue valves combine a tubular, or annular, structure (referred to as a “tubular structure” elsewhere herein) and two or more leaflets, the term used to describe cusps in such valves. In most of these valves, the tubular structure is typically made of synthetic materials, but can also be made of tissue or a combination of tissue and synthetic material. The tubular structure's role is to hold the leaflets and to connect the valve to the wall of the aorta and/or the heart. The leaflets are most often made of biological materials attached to the tubular structure and form the cusps or leaflet of the valve, which open to allow blood to flow in one direction, and close to prevent blood from flowing back in the reverse direction. In many cases, the tubular structure is provided by a collapsible stent, onto which are attached the leaflets.

The biological material used to make the leaflets of a tissue valve or valve that combines biological and synthetic components is typically pericardium sourced from cadavers or animals (e.g., of bovine, equine, or porcine origin). These xenogeneic materials are most always chemically or physically denatured (a process often referred to as “fixation”) to minimize the immune response of the recipient against what the body regards as a foreign material, and to thereby limit the degradative effect of this immune response to the valve once implanted. Fixation is also used to strengthen or otherwise change the mechanical properties of the biological tissue. Fixed tissues are still recognized by the body as foreign material, however. For example, fixed pericardium can trigger immune reactions or calcifications that lead to valve degradation and failure.

Some valve implants are made from whole animal valves (such as from the heart or a vein of an animal). The animal valves are also fixed to reduce immune responses. Fixation can promote calcification that leads to valve failure, however, so remains an imperfect solution. Sometimes, valves come from human tissue donors (i.e., are allogeneic or homograft) but these are in limited supply.

A number of different types of valve implants are therefore in use. Variations are provided by the form and materials of the tubular structure to which the leaflets attach. In some cases, it is possible to combine textile ‘skirts’ with the other materials to function as the tubular portion. A number of different designs are in use. In one, ‘funnel’-shaped, structure there are just leaflets attached to a stent. In another common combination, three leaflets and a synthetic (PTFE) tube are contained within a stent. In a third embodiment, an animal (e.g., bovine) valve, which has been cleaned and fixed, is attached to the inside of stent; there may also be a tube on the inside of the stent. In still other variations, a wire frame provides a support to which leaflets and a tube are sutured.

Fitting a patient with a heart valve is typically via open heart surgery, or via a minimally invasive procedure referred to as transcatheter aortic-valve implantation (TAVI), which uses collapsed valves around a delivery catheter. There are a number of clear advantages to minimally invasive vascular repair techniques, including shorter hospital stays, decreased surgical trauma, and the observable trends toward lower mortality rate and/or fewer complications in certain patient populations. These advantages have driven rapid clinical adoption of transcatheter approaches. In the case of heart valve repair, TAVI significantly reduces mortality in some patient populations compared to the previously standard treatment by open-heart surgery (see, e.g., Leon, M. B., et al., “Transcatheter aortic-valve implantation for aortic stenosis in patients who cannot undergo surgery”, N. Engl. J. Med., (2010) Oct. 21; 363(17):1597-607. Epub 2010 Sep. 22). In TAVI, the implantation procedure involves accessing a mid-size artery, often includes performing balloon valvuloplasty (in order to destroy the existing valve), and then requires bringing the new valve through the access vascular path for deployment at or near the valve location, for example at the heart/aorta interface. TAVI is an example of but one minimally invasive approach, but other minimally invasive approaches (transapical, transcutaneous, etc.) that rely on a collapsible valve have been deployed, according to a patient's circumstance. TAVI can be used to replace aortic valves, pulmonary valves, tricuspid valves, mitral valves, or venous valves.

There remain complications associated with valves delivered by TAVI, however. Current delivery assemblies (the collapsed valve and the deployment catheter) are bulky and difficult to push through the relatively small blood vessels that lead to the larger blood vessels of the heart. It has also been found that these procedures are plagued by higher incidence of strokes and major vascular complications compared even to open heart surgery, mainly due to the bulkiness of the valve assembly. Consequently, the most pressing challenge in the design of devices for TAVI is to decrease the cross-sectional area of the delivery assembly. However, even if the (synthetic) tubular structures can be engineered to be as thin as possible, the biological components (e.g., animal pericardium), which are harvested and not manufactured, are only available in certain thickness ranges. While various processing techniques can be used to make thinner tissues, these processes can be challenging and may not produce acceptable material. Fixed pericardium can also trigger immune reactions or calcifications that lead to valve degradation and failure over time. Pericardium that has been fixed can also be stiff, have poor bending properties and can be hard to compress into a catheter. It is also susceptible to delamination during its service life in the valve.

Besides animal-based tissues, various structures from human based tissues have been engineered in recent years. It has been observed that, during culture in the presence of ascorbic compounds, adherent cells, such as fibroblasts, can lay down large amounts of extracellular matrix proteins between cells and on culture surfaces. As a result, cohesive pieces of living tissue, comprised of the living cells and the extracellular matrix proteins produced by the cells, can be detached from the culture support and used in surgical applications (see, e.g., U.S. Pat. No. 6,503,273).

Typical cell cultures last for days to a few weeks before the cultures are contaminated by microorganisms, cells die, detach from the culture surface, or contract into small aggregates, any one of which would prevent effective production of a viable structure. However, cohesive tissue sheets can be produced using fibroblast cultures lasting 4 to 5 weeks, as well as up to 7 weeks (see, e.g., L'Heureux, FASEB J., (1998), 12:47-56, and U.S. Pat. No. 7,112,218.) This approach went against the general thinking in tissue engineering at the time, which was to minimize culture time to reduce the risk of contamination, reduce overall production time, and thereby reduce cost.

The living structures formed by these culture methods can be used in the production of living and completely biological constructs such as blood vessels that have high mechanical strengths without the need for artificial or other exogenous scaffolding (see, e.g., L'Heureux, N., et al., “A completely biological tissue-engineered human blood vessel”, FASEB J., (1998), 12:47-56; L'Heureux, N., et al., “Human tissue-engineered blood vessels for adult arterial revascularization”, Nature Med., (2006) March; 12(3):361-5. Epub 2006 Feb. 19; and McAllister, et al., U.S. Pat. Nos. 7,166,464 and 7,112,218, all of which are incorporated herein by reference). The methods of making and using such tissue constructs are sometimes termed tissue engineering by self-assembly (“TESA”) (see, e.g., Peck, M., et al., “Tissue engineering by self-assembly”, Materials Today, 14, 218-224 (2011); Peck, M., et al., “The evolution of vascular tissue engineering and current state of the art”, Cells Tissues Organs, 195, 144-158, (2012), both of which are incorporated herein by reference), or just “tissue engineering”.

One major advantage of this approach is that the tissues are made of native (i.e., un-modified, un-fixed, un-denatured) extracellular matrix that is more compliant than synthetic materials or animal tissues that have been fixed. This native extracellular matrix is also advantageous because it can be remodeled by the body and can potentially grow with the patient. Also, the matrix will not initiate significant degradative immune responses since it is of human origin. In addition, the tissues can also be grown to contain living cells (autologous and/or allogeneic) to improve remodeling, immunological acceptance and/or its physiological functions.

Depending on the culture support, the resulting tissues can have various shapes and sizes (sheets, ribbons, threads, and particles, as described in, for example, U.S. Pat. Nos. 6,503,273 and 7,166,464, U.S. Pat. App. Pub. No. 2010-0189792, and in International Patent Application Publication No. WO2012/145756, all of which are incorporated herein by reference). The process of making tissue sheets in this way has been termed sheet-based tissue engineering (SBTE) when the basic building blocks that are formed are planar sheets of tissue. SBTE has been used to produce completely biological, living, autologous human blood vessels from tissue sheet cultures eight weeks old (see, e.g., L'Heureux, N., et al., Nature Med., (2006), 12(3):361-5). Using this approach, tissue-engineered blood vessels with mechanical properties similar to those of native blood vessels can be built in vitro without the addition of exogenous materials or synthetic scaffolds. These completely biological human grafts have been shown to be safe in humans with observation points up to three years (see, e.g., McAllister, T. N., et al., “Effectiveness of haemodialysis access with an autologous tissue-engineered vascular graft: a multicentre cohort study”, Lancet, (2009), 373:1440-6; Gamido, S., et al., “Haemodialysis access via tissue-engineered vascular graft”, Lancet, (2009), 374:201; L'Heureux, N., et al., “Tissue-engineered blood vessel for adult arterial revascularization”, N. Eng. J. Med., (2007), 357:1451-3).

Prior to the technology described herein, a viable valve made by tissue engineering has yet to be constructed. Valves that contain tissue-engineered parts have been the subject of research only, and have yet to be used in humans. Current methods of making tissue-engineered valves follow an approach that has been tried and tested in other structures, and involves combining a synthetic scaffold, which provides the prerequisite mechanical strength, with living cells. The most promising efforts have led to animal valves, i.e., produced with animal cells, that were implanted in the same specie of animal. (See, e.g., Sutherland, F. W., et al., “From stem cells to viable autologous semilunar heart valve”, Circulation, 111, 2783-2791 (2005); and Sodian, R., et al., “Early in vivo experience with tissue-engineered trileaflet heart valves” Circulation, 102, 11122-29, (2000), both of which are incorporated herein by reference.) These valves were implanted as pulmonary valve replacements probably because the low pressure in the pulmonary artery is a more forgiving environment than elsewhere.

Previous attempts at using tissue sheets to produce heart valves have relied on stacking multiple sheets together (see, for example, U.S. patent application Ser. Nos. 10/198,628 and 10/495,748, both of which are incorporated herein by reference). Sheets are stacked principally to achieve a greater mechanical strength than would normally be required for another structure such as a blood vessel. The culture period for each of the sheets before stacking was stated to be as little as three weeks (LaFrance, H., et al., U.S. patent application Ser. No. 10/198,628), consistent with typical tissue culture times formerly used in tissue engineering. However, use of multiple layers involves complex production processes, and tends to produce leaflets prone to delamination because the stacked sheets lack cohesion.

Accordingly, there is a need for a method of producing a valve, such as a heart valve, via tissue engineering that has sufficient mechanical strength and does not delaminate during use.

The discussion of the background herein is included to explain the context of the technology. This is not to be taken as an admission that any of the material referred to was published, known, or part of the common general knowledge as at the priority date of any of the claims found appended hereto.

Throughout the description and claims of the application the word “comprise” and variations thereof, such as “comprising” and “comprises”, is not intended to exclude other additives, components, integers or steps.

### SUMMARY

The instant disclosure addresses the repair of valves such as heart valves, in a patient. In particular, the disclosure comprises a single-layer tissue sheet having a puncture strength of 2 kgf to 6 kgf that can be used in the construction of a valve implant. The disclosure further comprises an method for making such a tissue sheet, as well as a method of making a valve implant that contains such a tissue sheet.

In the technology described herein, the culture time of the tissue sheets has been extended by 3 fold or more compared to the sheets previously produced. The feasibility of culturing sheets for over 24 weeks, is demonstrated, and that the strength of the sheets reaches unprecedented strength over such long culture times.

This technology described herein is not limited to valves introduced by minimally invasive techniques. It is to be understood that any of the valves implants described herein can be introduced into a patient via open surgical approaches.

The present disclosure provides for a single-layer tissue sheet having a puncture strength of 2 kgf to 6 kgf. The single-layer tissue sheet comprises adherent cells and extracellular matrix produced by the cells. The single-layer tissue sheet can be formed by culturing for a period between 25 and 52 weeks.

A single layer tissue sheet having a puncture strength of 2 kgf to 6 kgf can have one or more cell-synthesized threads incorporated into it.

A valve implant comprises a tubular structure, and two or more leaflets, connected to the tubular structure, wherein the two or more leaflets each comprise a single-layer tissue-engineered sheet having a puncture strength of 2 kgf to 6 kgf.

The single-layer tissue sheet can be folded on to itself one or more times.

The present disclosure includes a process for making a tissue-engineered valve implant, the process comprising: growing a tissue sheet having a puncture strength that exceeds 2 kgf; cutting two or more leaflets from the tissue sheet; and attaching the two or more leaflets to a stent, thereby creating a valve implant.

The present disclosure further includes a method for repairing a heart valve, the method comprising: collapsing a tissue-engineered heart valve on to a transcatheter; and delivering the tissue-engineered heart valve into place via a catheter, wherein the tissue engineered heart valve comprises two or more leaflets made form tissue-engineered sheets.

The present disclosure still further includes a process for making a single layer tissue sheet, the process comprising: culturing a population of cells, in a culture dish with one or more tissue control rods, for a period exceeding 20 weeks, to make a sheet comprising the cells and extracellular matrix produced by the cells; and during the period and after two weeks, seeding the sheet with a suspension of cells from the same cell line as the population of cells, wherein the seeding is carried out 2-6 times.

### BRIEF DESCRIPTION OF THE DRAWINGS

FIG. 1: Results are shown for two cell lines of normal human skin fibroblasts; in each case the USTS is the right-hand bar. In the graph on the left, the puncture strength of a sheet cultured for 8 weeks, as previously described, is compared to that of a USTS that is 25 weeks old, for each of two cell lines. The USTS shows a strength increase of 4.4- and 5.8-fold. In the graph on the right, the strength/thickness ratio of a sheet cultured for 8 weeks, as previously described is compared to that of a USTS that is 25 weeks old. The USTS shows a strength/thickness ratio increase of 2.7- and 3.7-fold.

FIG. 2: A largely dried USTS (bottom) cut according to a cutting guide (top) to create the three leaflets of a heart valve. Each semi-lunar leaflet is a single layer of tissue sheet. The drying process makes the sheet appear transparent. The cutting guide included a tissue allocation to create a nodule of Arantius (the location where the three leaflets meet). The semi-lunar leaflets are connected by a band of tissue that will be folded to create a thicker region for securely sewing the upper region of the leaflet to create the commissure. This is one example of many possible designs of leaflets that can be constructed according to methods and description herein.

FIG. 3: The same cut USTS from FIG. 2 sewn onto a synthetic tubular structure made from PTFE to create a tricuspid valve for demonstration purposes (not implantation). The USTS is hydrated and each cusp is made from a single layer of tissue-sheet. The leaflets are sewn next to each other to create a commissure where the leaflets originate, juxtaposed to the tubular structure. The free edges of the cusps meet to seal the vessel at the zone of coaptation.

FIG. 4: The valve of FIG. 3 under backpressure seen from the top (in vivo, this would be the view from the aorta). The backpressure has filled the cusps' sinuses and forced them to close. The coaptation of the leaflets is clearly visible. Additional tissue is available to create a nodule of Arantius or to allow for tissue retraction during remodeling in vitro or in vivo.

FIG. 5: Tissue engineered leaflets (bottom) made of a USTS folded to create the straight edge, with cell-synthesized threads positioned between the two layers to provide additional strength along the axis of maximal stress. Two designs are shown: in a natural design, the organization of the fibers is less symmetrical than in an engineered design. The USTS are not fused together. Native bovine heart valve leaflet is shown at top for comparison.

FIG. 6: Graph of force vs. strain obtained during a suture pullout test performed with a USTS allowed to contract to 60% its original length before crosslinking with 0.5% glutaraldehyde (triangles), and with a USTS that was not allowed to contract before fixation (‘X”).

FIG. 7: Suture pullout results for test performed on the same sample of thread-reinforced USTS. The sutures are pulled out from different locations and in different directions. Sutures 1 and 2 are being pulled out perpendicular to the threads, from the same distance inside the material. Suture 1 is pulled just from the tissue, without crossing a thread. Suture 2 requires 46% more force than suture 1 to pull out. Sutures 3 and 4 compare pulling a suture out from the tissue sheet vs. pulling longitudinally through a thread. Data shown is for n samples in each suture configuration. Error bars are shown as raw numbers and percentages (e.g., for suture 1, ±11 gf and 15%).

Like reference symbols in the various drawings indicate like elements.

### DETAILED DESCRIPTION

The technology herein relates to valves for use in intravenous locations, or to replace heart valves, wherein one or more parts of the valves are made by tissue-engineering.

There are a number of advantages of applying sheet-based tissue engineering to valve replacement and repair. Robust biological tissue-sheets of controlled thickness are particularly well suited to produce valves for transcatheter delivery, since valves containing such tissue-engineered material have a cross-sectional area (often termed a “crossing profile”) that can be smaller than that of valves made with xenogeneic or cadaver tissues, such as pericardium. Because delivery of bulky items such as valves invariably causes some damage to an artery or vein when being positioned, a small sized object can offer significant clinical advantages. Because the tissue-engineered materials are manufactured through a controlled process, they also have the advantage of providing sheets of even thickness and homogenous structure compared to harvested tissues (e.g., they do not contain blood vessels, fat deposit, calcifications or other defects). This provides significant economic advantages by reducing the need for continuous quality control (QC) monitoring, and testing of the engineered tissue. In addition, the manufacturing process can guarantee the sterility of the starting material, something which cannot be done with, for example, material from a slaughterhouse. Since the tissue is engineered in vitro, it can also be prepared to fit specific needs for valve production (e.g., particular shape, variable thickness, and incorporate reinforcement structures where necessary) and be regionally tuned (reinforced) in various ways. Furthermore, tissue engineering also has the advantage of using human tissue in place of xenogeneic tissues, which can have immunological, remodeling and commercial advantages. Irrespective of valve design or application, leaflets made of human tissue would not be aggressively rejected by the immune system and can be positively remodeled and integrated into the surrounding tissues of the recipient. This improves long-term function of the valve by avoiding calcification and structural degradation. Because the tissue is human, it does not require chemical fixation to resist biodegradation mechanisms. An unfixed human tissue will be less likely to calcify than fixed biological grafts, have better mechanical properties (including flexibility), be less thrombogenic, will have growth potential, and can integrate in the surrounding tissue to achieve long-term stability. In a design where the tubular structure is also made of a completely biological tissue-engineered material, the lack of immune response and positive remodeling would increase the likelihood of better outcomes. Finally, a human product would be more appealing to patients than would an animal tissue.

Methods for producing a tissue-sheet suitable for making a blood vessel have been described elsewhere, see, e.g., U.S. Pat. Nos. 7,166,464, and 6,503,273, incorporated herein by reference. By using adherent cells such as fibroblasts seeded onto a cell culture substrate and grown for culture periods of several weeks in vitro in the presence of ascorbate compounds, a robust sheet can be formed. Sheets cultured for 4 to 8 weeks have been found sufficient to produce tissue-engineered blood vessels (see, e.g., L'Heureux et al., FASEB J., 12:47-56 (1998); and L'Heureux et al., Nature Med. (2006) 12(3):361-5). Once the sheet acquires strength sufficient for the desired application, it can be suitably manipulated.

In initial work using human tissue-sheets, they were typically cultured for 4 to 5 weeks (see, e.g., L'Heureux, et al., FASEB J., 12:47-56 (1998)). In later studies and in ensuing clinical trials, sheets typically cultured for 8 weeks were used (see, e.g., L'Heureux, et al., Nature Med. (2006) 12(3):361-5, McAllister, et al., Lancet, (2009), 373:1440-6; Gamido, et al., Lancet, (2009), 374:201; and L'Heureux, et al., N. Eng. J. Med., (2007), 357:1451-3).

### Ultra-Strong Tissue Sheets

The technology herein, in which tissue-engineering is deployed to make valves, has been made possible by the fact that cells can in fact be cultured for very long periods in sterile conditions, much longer than had been previously contemplated, to form tissue sheet referred to herein as an ultra-strong tissue sheet (USTS). Periods of culture can therefore extend from 4 months to 12 months. Other periods of culture for producing tissue engineered materials suitable for making valves include: 20 weeks, 24 weeks, 25 weeks, and periods in the ranges 25-30 weeks, 30-40 weeks, and 40-52 weeks. Periods may also be expressed in days, such as: 120-150 days; 135-145 days; 160-170 days; 170-180 days; 200-220 days; 210-280 days; 280-350 days; and 300-365 days. It can be assumed that the lower and upper end-points of each of these ranges can be interchanged with any other quoted end-point to provide alternative ranges of culture times (e.g., 210-300 days) fully described herein. A tissue sheet produced by the methods of the present invention is referred to herein as a USTS. It is a tissue sheet having characteristics of high strength and comprises a single layer of tissue.

The strength of the USTS can be further improved by serial seeding of cells during the culture period. A suspension of cells, typically from the same cell line from which the sheet is growing, can be seeded over the developing sheet. The cells can be seeded at the same density as that which was used to start the culture or at much higher densities such 5-fold, 10-fold, or 20-fold the starting concentration. This additional seeding can be performed at any time during culture but is typically performed from 2 weeks after the beginning of the culture and before 4 weeks before the end of the culture. This additional seeding can be performed many times during the culture period such as 2 to 6 times but preferably 2 to 3 times.

Culturing can take place in a bioreactor, for example as described in U.S. Pat. No. 7,744,526. Culturing can also be assisted by use of one or more objects referred to variously as tissue control rods or tissue manipulation devices, or other similar mechanical devices that can facilitate sheet formation in the bioreactor, anchor the sheet to prevent unintended sheet detachment or contraction, or assist manipulation of the sheets after removal from the bioreactor. Suitable mechanical devices include metal clamps, for example in L-shaped (including right angle) configurations, as well a continuous loop of metal such as a ring. Where two or more clamps are used to confine the growing tissue sheet, it is preferable to prevent the clamps from overlapping with one another as and when the tissue sheet contracts. For example, some form of mechanical constraint can be deployed to keep the two more clamps from moving significantly from their initial configuration, thereby keeping the tissue sheet taut. Due to the long culture times, and to avoid risk of contamination during that time, the bioreactor is typically a culture flask that can be closed, or which has a narrow opening at the top.

The tissue-sheets that can be produced in vitro by using the extended culture times described herein have mechanical strengths that surpass that of any other in vitro produced tissue-sheets previously described. It had not been previously understood that such prolonged culture times would lead to enhanced mechanical strength of the resulting tissue. For example, the mechanical strength of a rolled or layered structure formed from multiple sheets, or multiple layers of a sheet, does not increase after 7 weeks of culture time (see, e.g., L'Heureux et al., FASEB J., 12:47-56 (1998) and U.S. patent application Ser. No. 10/198,628).

In order to successfully produce such a mechanically strong tissue-engineered structure, as understood by one skilled in the art, a high degree of supervision and management of the tissue culture apparatus is required, mainly to avoid the possibility of contamination during the culture period. From such conditions, very strong tissues composed of a single homogeneous layer of tissue can be created, instead of relying on stacking or fusion methods that use multiple sheets of tissues cultured for shorter periods to create a composite tissue.

Hitherto, processes that involve a combination of stacking-and-fusion have been the standard methods to make mechanically strong structures with tissue-sheets. (See, e.g., L'Heureux et al., FASEB J., 12:47-56 (1998); L'Heureux et al., N. Eng. J. Med. (2007), 357:1451-3; Michel, M., et al., “Characterization of a new tissue-engineered human skin equivalent with hair”, In Vitro Cell Dev. Biol.-Anim., (1999), 35:318-26; L'Heureux, N., et al., “A human tissue-engineered vascular media: a new model for pharmacological studies of contractile responses”, FASEB J., (2001); 15:515-24.; Haraguchi, Y., et al., “Regenerative therapies using cell sheet-based tissue engineering for cardiac disease”, Cardiol. Res. Pract., (2011):845170, all of which are incorporated herein by reference.) These processes have several drawbacks: 1) They create tissues that may be strong, but are thick and therefore difficult to position intravenously; 2) they rely on the fusion of the sheets together (a lengthy, unpredictable and often incomplete process that relies on cell activity as well as applied forces); and most importantly 3) the resulting stack of tissue sheets is prone to delamination in an environment as harsh as that in which a heart valve operates.

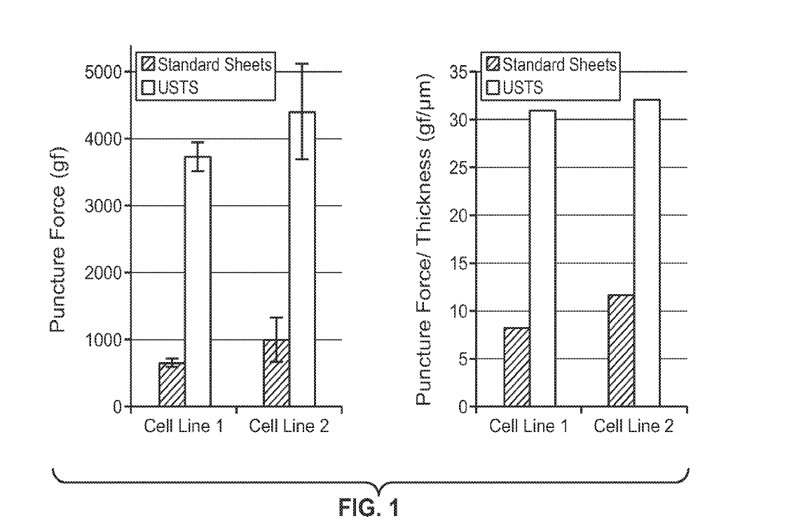

Sheet strength is often assessed by determining the force needed to puncture the sheet with a round-headed piston. It has been previously reported that sheet puncture strength can reach up to 800-1,000 gf with a piston head of 8-10 mm (see, e.g., L'Heureux et al., Nature Med. (2006) 12(3):361-5; U.S. Pat. Nos. 7,166,464; 7,504,258, and 8,076,137; and Peck, M., et al., “Tissue engineering by self-assembly” Materials Today, 14, 218-224, (2011)). According to this method of measurement, the measured puncture strength varies according to the size of the tool used. The values quoted herein are obtained when measured with a round-head piston (ball), 9.6 mm diameter, and driven through a tissue sheet secured with a clamping device to provide an exposed circular segment of the sheet having 25 mm in diameter. The measured value might be lower when measured with a smaller tip, e.g., a 1 mm tip. Through the prolonged cell culture techniques described herein, it can be demonstrated that sheet puncture strength can reproducibly reach 2 kgf and often exceed 4 kgf (FIG. 1), and can still further exceed 5 kgf. It is consistent with the methods described herein that a USTS can be made with a puncture strength of between 5 and 6 kgf when measured with a 10 mm ball. This represents approximately a 2.5-5-fold increase in strength over those tissues cultured for shorter periods of time as described elsewhere in the art. The fact that a completely biological sheet derived solely from cultured cells can reach such strength without any exogenous scaffold or without chemical or physical modifications is very unexpected, has not been shown before, and is contrary to the conventional thinking in the field hitherto. It is this unanticipated strength that allows creation of remarkably strong tissue using a single layer of USTS when others had assumed that a fused multi-layer of tissue-sheets would be needed.

The thickness of the USTS described herein can be difficult to measure precisely because the tissue is compressible and becomes thinner when, e.g., it is clamped in an attempt to measure its thickness. Nevertheless, thicknesses in the range: 200-400 microns are reasonable for a valve leaflet. Thicker sheets, say, up to 500-600 microns are also possible, as are thinner tissues, having thicknesses, down to 150 microns. Generally the thickness of the resulting USTS is controlled by length of culture time.

Furthermore, the USTS produced by methods described herein is not only surprisingly stronger than sheets previously described in the art, it also has a strength-to-thickness ratio that is approximately 300% higher than those sheets (see FIG. 1). This is of critical importance for the design of a heart valve that has a reduced cross-section suitable for use in TAVI. Since a goal of the technology is to reduce the crossing profile (diameter) of the valves, the use of a leaflet material that has a better strength/thickness ratio (i.e., is thinner but gives the same strength as materials previously used) is a good way to achieve that.

The unexpected strength of a USTS allows a single-layer leaflet design to favorably compare with native tissue. To describe the strength of a material, engineers use a quantity called ultimate tensile strength (UTS, or “stress”). This quantity is a “material property” or an “intrinsic” quantity, i.e., it is size-independent, and describes the force needed to break a material per unit of cross sectional area in a uniaxial tensile test. Native human aortic valve leaflets have a reported maximum UTS of 2.6±1.2 MPa. (See, e.g., Balguid, A., Rubbens, M. P., Mol, A., et al., “The role of collagen cross-links in biomechanical behavior of human aortic heart valve leaflets—relevance for tissue engineering”, Tissue Eng., 2007; 13:1501-11.) The UTS of the USTS described herein has been measured to be around 4.7±0.6 MPa without any chemical or physical modifications. Preliminary data have shown that this strength can be increased by a further 30% using simple formaldehyde fixation. Also, the USTS is highly compressible due to its high water content. Using a compressed thickness, its UTS is increased by approximately 3-fold. Again, this remarkably high strength “density” makes the USTS uniquely suited to tissue-engineered applications that require a thin and cohesive structure that is also very strong, such as for valves and especially valves designed for TAVI. Xenogeneic bovine pericardium has a UTS that is about 2-fold that of compressed USTS, but such high strength is likely not needed for leaflet production considering the UTS of native tissue (see, e.g., Vincentelli, A., et al., J. Heart Valve Dis. 1998; 7:24-9) Currently significant effort is required in selecting the right pericardium for TAVI applications. Both calf and porcine pericardium have been used to date. The specific material may be chosen by identifying the thinnest spots in the whole pericardium sac. The pericardium may be a stronger material per unit thickness than a tissue-engineered sheet, but the pericardium is not available in any thickness and therefore requires a lot of quality control in selecting an appropriate sample.

A single sheet design also has advantages for valve creation. The unique ability to create ultra-strong tissue sheets allows tissue engineering to address applications that could not be successfully addressed before. For example, in U.S. patent application Ser. No. 10/198,628, tissue-sheets produced in vitro for the purpose of making a heart valve relied on stacking-and-fusing at least five (up to nine) sheets per leaflet, although whether even this produced sufficient strength was never demonstrated. The sheets that formed the stack were only cultured for 3 weeks. There are no publications that describe the resulting performance of a valve constructed in this way; nor was any mechanical or functional data provided in the application. Because the methods herein do not rely on a stacking-and-fusion strategy to achieve adequate strength, a superior valve that will not be susceptible to delamination can be constructed. Even in biological valves made from fixed animal tissue, delamination of the leaflets has been an issue after implantation, suggesting that a stack of tissue-sheets would be particularly ill-suited for use in valves (see, e.g., Mirnajafi, A., Zubiate, B., and Sacks, M. S., “Effects of cyclic flexural fatigue on porcine bioprosthetic heart valve heterograft biomaterials,” J. Biomed. Mater. Res., A 94, 205-213 (2010)). For example, it is found that the leaflets of a native porcine valve are made of 3 layers (two thicker stronger layers sandwiching a weaker layer in the middle). Although naturally the valve is not prone to delamination during the animal's lifetime, fixing the valve for use in human applications stiffens it, remove some cellular components, and thereby weakens it

Once formed, a tissue engineered sheet having sufficient strength can be cut into the shape of a valve component, such as a leaflet. In a preferred embodiment, a single USTS is produced in vitro and used to create each leaflet of the valve. One skilled in the art of tissue engineering will acknowledge that there are numerous valve designs, for TAVI or other applications, where leaflets can be formed by such a USTS. The tissue sheet described herein can be used to create components for synthetic valves to replace any type of heart valve, including: aortic valves, pulmonary valves, tricuspid valves, mitral valves, or venous valves. Synthetic valves using the tissue sheets herein can be designed to be collapsible and delivered by means of a catheter, or designed to be delivered by open surgical techniques. The structure to which the leaflets are attached can be a collapsible stent, a self-expanding stent, or a non-collapsible frame made of metal wire and/or of a biocompatible polymer. This structure can also be made of a biodegradable polymer, a tissue sheet, another USTS, or cell-synthesized threads (for example, as described in U.S. Pat. App. Pub. No. 2010/0189792). The structure to which the tissue sheets are attached can perform a dual role as part of a delivery device (e.g., a stent) as well as part of the tubular portion of the valve. In some instances, a stent may also provide a spring-like structure to assist in the opening and closing of the valve.

USTS are much better suited for creating valves for TAVI than are sheets previously made, because valves used in TAVI are tightly folded when collapsed and deployed, a process which could therefore easily delaminate a laminated sheet made from a fused stack. In addition, the single USTS described herein has a higher strength per unit of thickness than any previously reported tissue-sheets produced in vitro. As a result, the USTS can create a thinner leaflet with the same or greater mechanical strength as those produced by stacking multiple thinner sheets. Consequently, a valve for TAVI can be produced with a smaller crossing profile than with stacked sheets (or with animal, e.g., bovine, pericardium). One skilled in the field of tissue engineering will acknowledge that providing a smaller crossing profile for a valve assembly is one of the most, if not the most, critical efforts in developing improved, next generation technologies.

### Valve Design

One skilled in the art will recognize that USTS are well-suited for many different valve designs. While a USTS can be constructed, as described elsewhere herein, without a stacking-and-fusion step, there may be designs of valves that can use a tissue sheet that has been folded upon itself to create two, or up to four, layers of tissue. In this instance, because of the intrinsic strength of the USTS, these layers of tissue do not need to be fused to one another, such as by the action of the cells (for example as previously described by LaFrance et al., U.S. patent application Ser. No. 10/198,628), or by compression (for example as described in U.S. Pat. No. 7,521,431). In some designs, the portions of the USTS can be glued together using an exogenous adhesive. In some designs, the portions of the USTS can be joined together with suture material or other types of threads, surgical clips, laser welding, or other methods known in the art. A multilayer of tissue may be needed for many reasons including, but not limited to: 1) wrapping the USTS over a stent or other non-biological components of the valve; 2) increasing the mechanical strength of the leaflet, 3) creating a thicker or stronger region to more easily or effectively suture the USTS to itself or another component of the valve, and 4) to create the tubular structure of the valve.

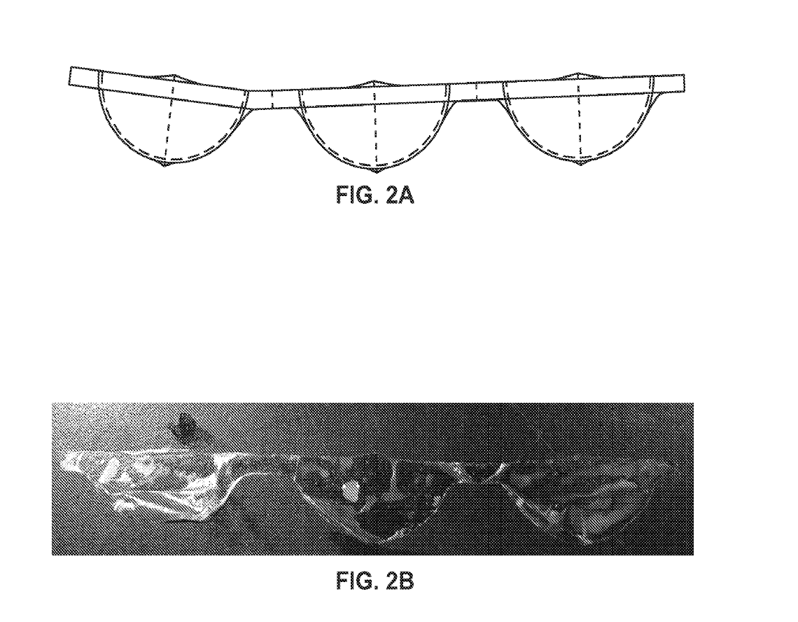

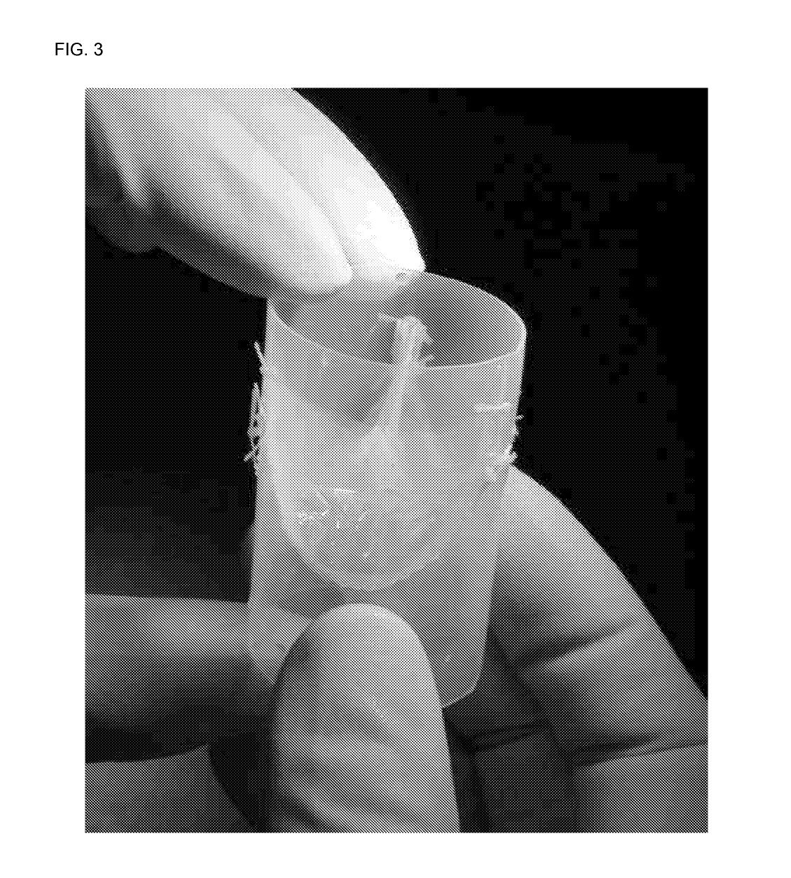

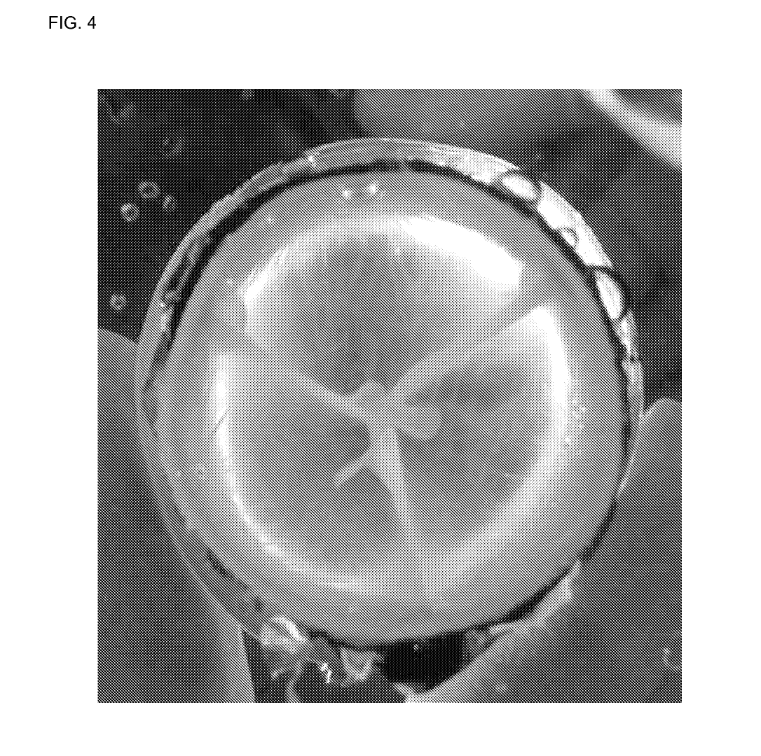

The USTS may also be gathered, wrinkled or pleated, to create regional changes in tissue density, independent of stacking layers of sheet, for various reasons, including adding mechanical strength, improving suturing strength or ease, improving coaptation, or improving hemodynamics. (See FIGS. 2-4) Coaptation is the name given to the process whereby the valve leaflets close upon one another during valve operation. When blood flows in one direction, the leaflets of valve open up; when the blood pressure decreases, the line where the leaflets seal to prevent backflow of blood is the coaptation line.

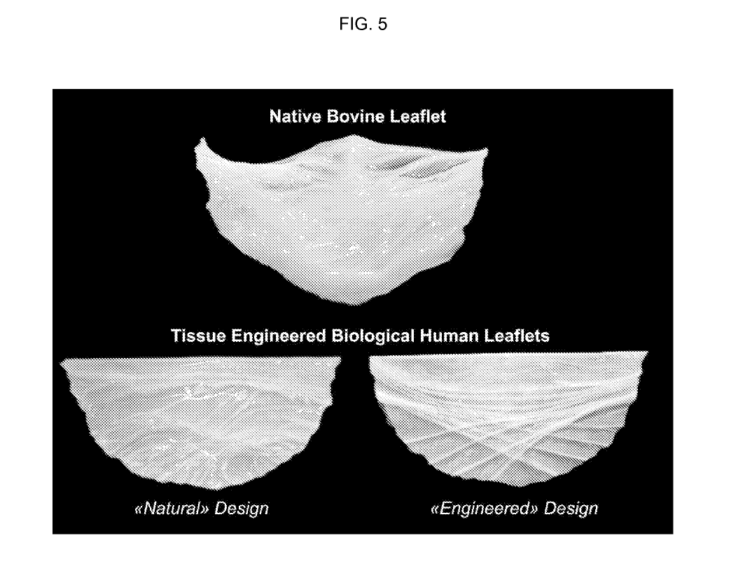

Valves that incorporate a USTS can also include tissue-engineered cell-synthesized threads. Examples of such threads are described in U.S. Pat. App. Pub. No. 2010-0189792. These threads can be added to directionally and regionally reinforce the USTS to create stronger leaflets, to sew one or more USTS together, or to sew leaflets to the other various components such as a vascular stent or the tubular structure of the valve (see FIG. 5).

The valve can be created entirely from biological tissues produced in vitro from any combinations of two or more of the following: tissue-sheets as produced by methods described elsewhere, e.g., U.S. Pat. No. 6,503,273, USTS as described herein, cell-synthesized threads, and cells seeded on to the valve material(s) to cover them, or cells placed in between layers of tissue material to seed a regenerative cell population (e.g., by introducing bone marrow cells inside layers to repopulate it). Examples of suitable regenerative cells for this purpose include but are not limited to: myofibroblasts, myocytes or their precursors, smooth muscle cells or their precursors, macrophages, mesenchymal stem cells, fat-derived stem cells, induced pluripotent cells, various types of undifferentiated cells, or a combination of any of the foregoing cell types. Valves created in this way can have significant advantages over implants made from cadaver tissue, non-human tissues, or synthetic materials, or from combinations of those materials. The valve can also be comprised of a USTS combined with any other existing valve prosthesis where one or more leaflets are replaced, reinforced, or repaired by a USTS. Valves can be assembled using the USTS utilizing any methods known in the art that uses a sheet of tissue, such as a pericardium, as a starting material.

While the fibroblast is the preferred cell type for USTS production, such sheets can be produced using other cell types such as, but not limited to: myofibroblasts, myocytes or their precursors, smooth muscle cells or their precursors, macrophages, mesenchymal stem cells, fat-derived stem cells, induced pluripotent cells, various types of undifferentiated cells, or a combination of any of the foregoing cell types. The cell lines used can be finite cell lines, semi-continuous cell lines or continuous cell lines. Certain stem cells may have key advantages in terms of immunogenicity. However, the method of sheet-based tissue engineering as described herein is not limited to particular cell type(s). Any cell type, or combination of cell types, that produces a sheet with adequate strength can be used. In addition, cells that do not produce a USTS on their own can also be mixed with USTS producing cells to provide desired advantages such as, but not limited to, accelerated USTS production, improved mechanical properties, immunocompatibility, providing metabolic or secretory activity, adding blood compatibility (for example by placing endothelial cells on the sheets to avoid blood coagulation on the sheet material), and limiting thrombogenicity, or favoring in vivo tissue integration, remodeling or performance.

The cells from which a USTS are made can be from the patient (autologous), from a donor (allogeneic), or from an animal (xenogeneic). The sheets can also utilize cells that have been genetically modified to: secrete specific factors, proliferate faster or longer, have better survival, have lower nutrient requirement, promote healing, treat a deficiency, cure a disease, or have other advantages. Of particular interest is the use of genetically modified cells to produce extracellular matrix (ECM) components in larger quantities than unmodified cells would typically produce. ECM components produced in this way can include, but are not limited to: collagen, elastin, laminin, fibronectin, vitronectin, tenascin, fibrilin, hyaluronic acid, chondroitin sulfate, dermatan sulfate, heparin sulfate, keratin sulfate, and versican. Also, the cells can be modified to produce matrices that are not naturally produced by the cells but are produced by other cell types, or by other living organisms (e.g., silk, chitosan, cellulose). Finally, cells can be genetically modified to produce an ECM component that is not found in nature, for example an ECM molecule with a modified amino acid sequence or of a length not normally found in nature.

Furthermore, the sheets can be cultured in the presence of exogenous elements that become part of the sheet and, ultimately, the resulting leaflet or valve. These elements can be added at any time during the growth of the sheet. The elements include structures such as protein aggregates, natural or synthetic fibers such as synthetic sutures or cat-gut, and mineral, and plastic or metallic devices. They can include items such as: needles, anastomotic devices, drug delivery devices, and magnetic or electronic devices such as radio frequency ID tags. Besides offering ways of identification, these devices can serve to facilitate or enhance further manipulation, mechanical strength, storage, surgical use or healing of structures that are based on USTS. Of special interest is the inclusion, by growing into the sheet, of cell-synthesized threads (or ribbons), as described in application no. PCT/US07/85148.

Typically, the sheets are obtained by growing adherent cells on a permissive substrate in a culture medium that contains salts, sugars, lipids, proteins, growth factors and an ascorbate compound, in a 5%:95% CO₂: air mixture, at 37° C. A multitude of culture conditions can lead to the formation of a USTS including, but not limited to: the use of animal or human serum or serum extracts as a source of proteins, lipids, growth factors or other biological macromolecules, which can improve cell growth and/or ECM production; the use of synthetic, purified or recombinant growth factors, lipids, proteins, sugars or other biological macromolecules, which may also improve cell growth; the use of so-called “serum-free” media, which has important regulatory advantages; the use of lower or higher concentration of oxygen, of CO₂ or other gasses; the use of temperatures between 25° C. and 45° C.; the use of culture media with pH ranging from 4.0 to 10.0; the use of culture substrates with patterned surfaces to improve cell attachment, cell growth, cell orientation, cell phenotype, extracellular matrix deposition, or to create regional differences in the sheet; the use of a coated culture substrate to improve cell attachment, cell detachment, cell growth, cell orientation, cell phenotype, extracellular matrix deposition, to create regional differences in the sheet or to select for specific cells; the use of a culture surface with a specific geometry to facilitate valve creation or to create regional differences in sheets thickness, strength, composition of organization.

### Sheet and Valve Treatments:

Since the technology described herein does not rely on a stacking-and-fusion strategy, the USTS does not need to be living at the stage that it is incorporated into a valve assembly. Accordingly, the USTS can be devitalized before, after, or at any step of the assembly process. The devitalization can be complete or partial, and it can be achieved by one or more methods known in the art including, but not limited to: drying; heating; cooling; freezing; adding compounds such as acids, enzymes, antibodies, detergents, salts, toxins or solvents; or applying various forms of energy, including, but not limited to ultrasound, electromagnetic or particle-based irradiation, mechanical forces such as centrifugation, fluid flow, and osmotic pressure. The devitalization can have other effects, or be performed for purposes other than devitalization itself such as, but not limited to: increasing mechanical strength; reducing immunogenicity; or improving implantation outcome.

In addition or alternatively, the USTS can also be decellularized (i.e., to remove cellular debris). It can be decellularized before, after, or at any step of the assembly process. The decellularization can be complete or partial. The decellularization can be achieved by any method known in the art such as, but not limited to, the use of solvents, acids, chemicals, enzymes, detergents, osmotic pressure or any combination thereof, including repeated treatments using the same method. This process can be improved with various mechanical treatments, fluid perfusion or exposure to various sources of energy, or any combination thereof.

In addition to, or alternatively, before, after, or at any step of the assembly process, the USTS or resulting valve can be coated with biological agents such as, but not limited to, exogenous extracellular matrix proteins, anti-platelet or anti-thrombogenic agents, natural or recombinant DNA or RNA, transfection agents, antibodies, growth factors, antibiotics, anti-proliferation agents, or a fragment thereof, or any combination thereof. The USTS, or resulting valve, can also be seeded with new cells before, after, or at any step of the assembly process. The cells can be of the same or of a different type than the ones used for the USTS production, or a combination of cell populations. These cells can also be cells that would not ordinarily form a USTS on their own, such as endothelial cells, mesothelial cells, keratinocytes, neurons, glial cells, islet cells, hepatocytes, or other cells that would provide a desirable advantage. These cells can be from the patient (autologous), non-autologous human cells (allogeneic), animal cells (xenogeneic), genetically modified cells (human or animal), or any combination thereof.

One skilled in the art will further understand that fixation can be performed with a wide array of chemical reagents, under various static or dynamic conditions (such as pressure, temperature, perfusion, tension, compression), and for various durations.

In addition or alternatively, the USTS, or resulting valve, can be treated with more or less powerful cross-linking agents including aldehydes, which can be used to achieve a complete or partial devitalization, to reduce immunogenic effects, to retard biodegradation, to modify mechanical properties or to attach chemical or biological compounds. The USTS can be cross-linked before, after or at any step of the assembly process. It can be cross-linked under mechanical stress to achieve desired mechanical properties, to achieve desired physical dimensions, or to improve implantation, healing, functionality or other desirable properties.

The living USTS can be allowed to contract during the culturing process, in order to improve one or more of its mechanical properties. One such property is compliance (the opposite of stiffness) or elasticity, which can be important to facilitate valve assembly as well as improve valve functionality. In addition, suture retention strength (the ability of a material to resist suture pull out, i.e., the ripping out of a suture stitch.) can also be improved by allowing contraction. The USTS can be allowed to contract in culture by cutting it free from any tissue manipulation devices, such as L-clamps, for example by disengaging interlocking L-clamps or by cutting the L-clamps away (the use of L-clamps is described in Example 1 hereinbelow). Contraction can start at any time during USTS production and can be allowed to proceed in culture for periods of time including days to weeks. Contraction can also be constrained by securing the sheet's edges at a set dimension. Contraction can also be restricted to a specific axis or direction in the sheet. Any design of clamps with dimensions that can be changed during sheet culture in a controlled fashion can be used. A complex pattern of contraction can be allowed to regionally tune the sheets' mechanical properties and fiber orientation. This regional tuning can be advantageous to improve suture retention strength. Additionally, anisotropic mechanical properties can be intentionally created to improve leaflet performance or to mimic native tissue biomechanics. The culture process can be continued after contraction has occurred. Contraction can be achieved in multiple steps where the clamps are brought closer after various time intervals. Some steps can include increasing the clamp distance, effectively stretching the sheet. Sheets cross-linked after or during contraction are of particular interest to provide a resilient tissue with advantageous mechanical properties such as elasticity and strength.

The living USTS can also be mechanically conditioned by dynamically applying force to the sheet during the culture process. One skilled in the art will understand that there are many methods to apply dynamic forces, that these forces can be applied with various amplitudes and frequencies, and that these forces can be applied in various directions. Many different types of bioreactors can be used for that purpose.

### Other Uses of USTS

The unique strength of a USTS can also be useful in other applications such as venous valves, coated vascular stents, endovascular grafts, vascular graft, vascular patches, heart patches, hernia patches, orthopedic patches, soft tissue repair or replacement, septum repair devices, ligament or tendon repair or replacement, skin wound repair or replacement, or any of the many other health-related applications where a sheet of material (biological or synthetic) is currently used.

### EXAMPLES

### Example 1

### Valve Formed from a Living Tissue USTS

This example describes one approach to creating a valve using a USTS. In this example, the resulting valve is a living tissue, which precludes using a terminal sterilization, or fixation, step. Accordingly, all of the assembly steps are performed in a sterile environment, with sterile liquids and instruments. This description is not intended to limit the scope of this invention with regards to the production method of the USTS, cell types, cell source, cell age, cell line, culture conditions, shape of the leaflets, leaflet numbers, suture material, suture method, type of valve frame or intended use of the valve. One skilled in the art can readily appreciate that various modifications can be made to the method without departing from the scope and spirit of the invention.

Typically, the sheets are obtained by growing normal human skin fibroblasts in T-225 cm² flasks. Cells are seeded at a density of 10,000 cells/cm² in DMEM supplemented with Ham F12 (20%), FetalClone bovine serum (20%), glutamine (2 mM), penicillin (100 U), streptomycin (100 mg/ml) and sodium ascorbate (500 mM) and cultured in a 5%:95% CO₂:air mixture, at 37° C. Spent media is changed for fresh media 3 times per week. After approximately one week, two L-shaped interlocking clamps made of 0.030 O.D. wire (304 stainless steel) are introduced in the flask. The two L-clamps effectively create a frame at the periphery of the flask that will be embedded in the USTS and effectively anchor it during culture. A frame external to the flask and positioned below it can be used to position magnets to secure the L-clamps in place. These clamps are tissue manipulation devices as described in U.S. Pat. No. 7,504,258, incorporated herein by reference. After a culture period of 25 weeks, the flask is cut open with a hot wire and the sheet is removed with the help of the embedded L-clamps. The wet sheet is laid on a cutting surface and 3 leaflets are cut with a die. The leaflets are kept wetted by culture medium at all times. The leaflets are sown onto a tubular stent (approximately 28 mm O.D.) using 6-0 PTFE sutures. The living valve is stored in culture media at 4° C. if the valve is to be implanted the same day. If implantation is performed later, the living valve is placed in a 5%:95% CO₂: Air mixture, at 37° C. The valve is crimped around a delivery balloon, sheeted and packaged sterilely. The valve is delivered to the target anatomical position following standard medical practice of interventional cardiology.

### Example 2

### Cryo-Preserved Valve

In this example, the valve is non-living. A USTS is produced as described in Example 1 but, at the end of the 25 week culture period, the culture medium is removed, the sheet is rinsed with sterile distilled water (WFI “Water For Injection”, Hyclone) and the flask is stored in a −80° C. freezer. This storage period can be from short periods (e.g., hours) to long (e.g., years). When needed, the flask is thawed and the sheet is used for valve assembly as described in Example 1. Because no special care is given to maintain cell viability during cryopreservation (e.g., no use of freezing media, no controlled freezing speed), only a small number, if any, of the cells will survive. The valve can be stored at 4° C. in a simple phosphate buffered saline for an extended period, until implantation. Such storage conditions will lead to all cell death. When ready for implantation in a patient, the valve is crimped around a delivery balloon, sheeted and packaged sterilely. The valve is delivered to the target anatomical position following standard medical practice of interventional cardiology.

### Example 3

### Dehydrated Valve

This example describes another method of making a non-living valve. A USTS is produced as described in Example 1, but at the end of the 25 week culture period, the culture medium is removed, the sheet is rinsed with sterile distilled water (WFI “Water For Injection”, Hyclone) and the sheet is dehydrated. The dehydrated sheet can be stored at room temperature, 4° C. or −80° C. for extended storage (hours to years). When needed, WFI is pipetted in the flask and the sheet is allowed to rehydrate (typically for 1 hour to 24 hours). The sheet is then used for valve assembly as described in Example 1. Normal human cells do not survive dehydration. The valve can be stored at 4° C. in a simple phosphate buffered saline for an extended period, until implantation. The valve is crimped around a delivery balloon, sheeted and packaged sterilely. The valve is delivered to the target anatomical position following standard medical practice of interventional cardiology.

### Example 4

### Sterilized Valve

In this embodiment, a non-living valve is assembled under non-sterile conditions and terminally sterilized. A USTS is produced using sterile techniques and stored sterilely as described in Examples 2 or 3, then the tissue is manipulated non-sterilely but still under cleanliness levels compatible with the production of a medical device for human implantation. This significantly simplifies the assembly process in part because the assembly can take place in a clean room with controlled air instead of in a laminar flow hood. Once the valve is assembled, the valve is crimped around a delivery balloon, sheeted and packaged. The final assembly is sterilized using gamma irradiation at a dose 25 kGy.

### Example 5

### Valve Leaflet from Folded USTS

In this embodiment, the shape of the leaflet and its mirror image are cut out of the USTS in a single piece. This piece is folded along the symmetry line (the imaginary mirror) to create the final leaflet. This leaflet is effectively made of two layers of USTS. The free edge of the leaflet is where the USTS is folded. This is an important characteristic of this method because the free edge is the edge of the leaflet that is most susceptible to delamination since the other edges will be sutured together and to the valve stent. This leaflet design does not require fusion of the two layers of the leaflet to create a functional valve. This leaflet design can be used to build valves according to any of the methods described in Examples 1 to 4.

### Example 6

### Three-Layered Valve Leaflet

In this example, a three-layer leaflet is produced. First, a piece of USTS is cut in the shape of the leaflet, and its mirror image as a single piece, as described in Example 5. Then, the shape of the leaflet is cut from the USTS and placed over the first piece, aligning the free edge of the leaflet with the symmetry line of the first piece. The leaflet covers exactly half the first piece. Then, the first piece is folded as described in Example 5. This leaflet design does not require fusion of the three layers of the leaflet to create a functional valve. This leaflet design can be used to build valves according to any of the methods described in Examples 1 to 4.

### Example 7

### Contracted USTS

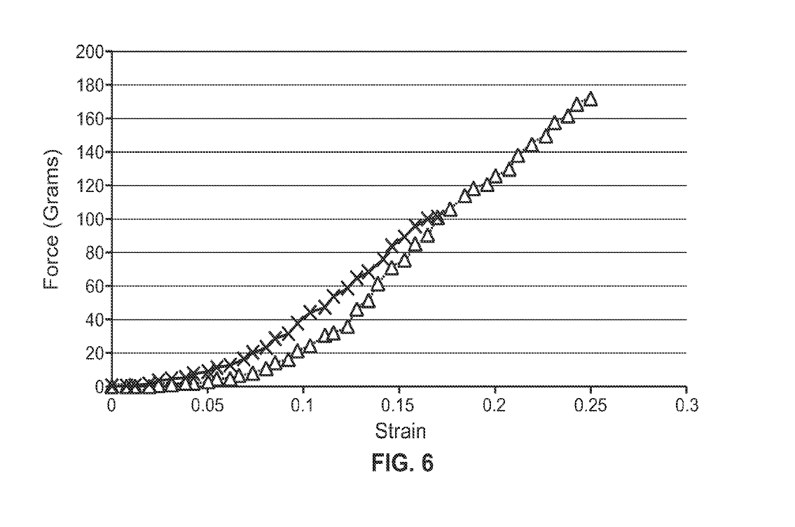

In this example, the USTS is allowed to contract in culture by cutting it free from the L-clamps and by reattaching two opposite edges at a set distance, thereby allowing a limited level of contraction in one direction. For example, a 28-week-old USTS can be released from its L-clamps and allowed to contract in culture for 7 days before being fixed in 0.5% glutaraldehyde solution for 24 hours (to provide cross linking). In FIG. 6, are shown the results of a suture pullout test that compared the behavior of USTS contracted with USTS not contracted, prior to glutaraldehyde fixation. Two results can be observed. First, the contracted tissue is more compliant since it will stretch more than the non-contracted tissue for a given force (up to 20% in this case). Second, the contracted tissue is more resistant to suture pullout (higher suture retention) since the ultimate force needed to pullout the suture is much higher than that of the non-contracted sheet (by 70%).

### Example 8

### Cell-Synthesized Threads Combined with a USTS

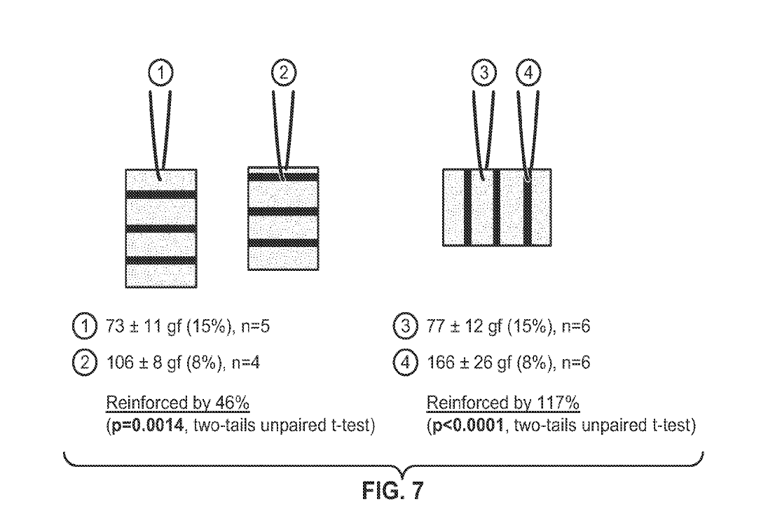

This example demonstrates how cell-synthesized threads can be used to improve leaflet production by combining them with a USTS. Cell-synthesized threads are textile-like products constructed from the extracellular matrix produced by cultured human fibroblasts (as described in U.S. patent application Ser. No. 12/515,397). These threads can be produced in a variety of sizes and strengths, and can be incorporated into a valve leaflet design to mimic the thick collagen fibers macroscopically observable in native heart valve cusps (see FIG. 5). Strategic positioning of these threads can transfer some of the mechanical loads directly to the valve frame (i.e., the stent for a collapsible valve, or a ring-like or tube-like structure made of various polymers/metal/textiles/tissues for the non-collapsible ones). In one experiment, cell-synthesized threads were closely positioned onto a sheet at 15 weeks, and the structure was further cultured for 10 weeks. At that point, the threads were embedded in the USTS and the tissue was fixed in glutaraldehyde (−1%). FIG. 7 shows the results of suture pullout experiments, demonstrating that the inclusion of cell-synthesized threads in the USTS can improve suture retention strength by from 46% to 117% depending on thread orientation. Alternatively, threads can be stitched in the USTS to provide support without the need for embedding. The threads can also be used as a suture material to assemble the leaflets to the valve frame or stent, or to sew leaflets together.

### Example 9

In this example, cell-synthesized threads are combined with a two-layer leaflet as produced in Example 5. Cells-synthesized threads can be placed between the two layers of USTS and roughly parallel to the free edge to provide circumferential support. Various arrangements are possible (FIG. 5). The ends of the cells-synthesized threads are fixed on the valve frame or stent. This can be achieved by tying the thread to the frame or stent, or by suturing the threads. In this embodiment, threads are not embedded in the USTS.

All references cited herein are incorporated by reference in their entireties.

The foregoing description is intended to illustrate various aspects of the instant technology. It is not intended that the examples presented herein limit the scope of the appended claims. The invention now being fully described, it will be apparent to one of ordinary skill in the art that many changes and modifications can be made thereto without departing from the spirit or scope of the appended claims.

What is claimed:

1. A single-layer tissue sheet having a puncture strength of 2 kgf to 6 kgf.

2. The single-layer tissue sheet of claim 1, comprising adherent cells and extracellular matrix produced by the cells.

3. The single-layer tissue sheet of claim 1, which has been formed by culturing for a period between 25 and 52 weeks.

4. The single-layer tissue sheet of claim 1, which has been formed by culturing for a period between 25 and 30 weeks.

5. The single-layer tissue sheet of claim 1, further comprising one or more tissue-engineered threads.

6. A valve implant comprising:
a tubular structure; and
two or more leaflets, connected to the tubular structure,
wherein the two or more leaflets each comprise a single-layer tissue-engineered sheet having a puncture strength of 2 kgf to 6 kgf.

7. The valve implant of claim 6, wherein the single-layer tissue sheet is folded onto itself so that each leaflet is comprised of two layers of tissue.

8. The valve implant of claim 6, further comprising one or more cell-synthesized threads.

9. The method of making a tissue-engineered valve implant, the method comprising:
growing a tissue sheet having a puncture strength that exceeds 2 kgf;
cutting two or more leaflets from the tissue sheet; and
attaching the two or more leaflets to a stent, thereby creating a valve implant.

10. The method of claim 9, wherein the growing comprises culturing the tissue sheet for a period between 20 and 52 weeks.

11. The method of claim 9, further comprising dehydrating the tissue sheet prior to cutting.

12. The method of claim 9, further comprising sterilizing the valve, after it has been formed.

13. The method of claim 9, further comprising contracting the sheet during growth.

14. A valve implant produced by the method of claim 9.

15. A method of repairing a heart valve, the method comprising:
collapsing a tissue-engineered heart valve on to a transcatheter; and
delivering the tissue-engineered heart valve into place via a catheter,
wherein the tissue engineered heart valve comprises two or more leaflets made form tissue-engineered sheets.

16. A method of making a single layer tissue sheet, the method comprising:
culturing a population of cells, in a culture dish with one or more tissue control rods, for a period exceeding 20 weeks, to make a sheet comprising the cells and extracellular matrix produced by the cells; and
during the period and after two weeks, seeding the sheet with a suspension of cells from the same cell line as the population of cells,
wherein the seeding is carried out 2-6 times.
